SEQUENCE LISTING (8 sequences)

SEQ ID NO: 1 - DNA, 23 bp, Artificial
  Primer
gcaaaaatacacttgtgggagaa

SEQ ID NO: 2 - DNA, 22 bp, Artificial
  Primer
ggtgctcagactccgacataat

SEQ ID NO: 3 - DNA, 20 bp, Artificial
  Primer
accaacgatggtgtgtccat

SEQ ID NO: 4 - DNA, 20 bp, Artificial
  Primer
cttgtcgaaccgcataccct

SEQ ID NO: 5 - DNA, 23 bp, Artificial
  Primer
gccaaaaaattgtttccacaata

SEQ ID NO: 6 - DNA, 25 bp, Artificial
  Primer
tcttcatcaccatacttttctgtta

SEQ ID NO: 7 - DNA, 27 bp, Artificial
  Primer
gttgttctataagctggtattgatgca

SEQ ID NO: 8 - DNA, 28 bp, Artificial
  Primer
ggaattcacatggtctactactgactgt

(19) United States
(12) Patent Application Publication
(10) Pub. No.: US 2009/0186346 A1
(43) Pub. Date: Jul. 23, 2009

(54) Method for Detecting the Presence or Absence of a Target Cell in a Sample

(75) Inventor: Marc Angles D'Auriac, Olso (NO)

Correspondence Address: CROWELL & MORING LLP;INTELLECTUAL PROPERTY GROUP, P.O. BOX 14300, WASHINGTON, DC, 20044-4300, US

(73) Assignee: GENPOINT AS, Oslo (NO)

(21) Appl. No.: 12/086,019

(22) PCT Filed: Dec. 12, 2006
(86) PCT No.: PCT/GB2006/004629

§ 371 Date: Dec. 15, 2008

(30) Foreign Application Priority Data
Dec. 12, 2005 (GB) 0525231.7

(51) Int. Cl. C12Q 1/68

(52) U.S. Cl. 435/6

(57) ABSTRACT

The present invention relates to a method for detecting the presence or absence of a target cell in a sample, said method comprising (a) binding cells in said sample to a particulate and mixable solid support; (b) eluting the cells from the solid support without the use of competitor molecules to disrupt the interaction between the cell and the solid support; (c) after lysis of said cells, detecting the presence or absence of nucleic acid characteristic of said target cell, wherein said solid support does not have antibodies or antibody fragments immobilised thereon. Kits for carrying out the method of the invention are also provided.

The present invention relates to a method for detecting the presence or absence of a target cell in a sample, in particular a method for detecting the presence or absence of a target bacterium in a sample which method comprises a nucleic acid based detection step.

Many of the methods for detecting cell types or microorganisms in a sample in use today rely on the identification of DNA or RNA e.g. diagnosis of microbial infections, forensic science, tissue and blood typing, detection of genetic variations etc.

The use of DNA or RNA identification is now widely accepted as a means of distinguishing between different cells or cell types or between variants of the same cell type containing DNA mutations. Thus, HLA typing, which is more commonly carried out by identification of characteristic surface antigens using antibodies, may alternatively be effected by identification of the DNA coding for such antigens. Microbial infection or contamination may be identified by nucleic acid analysis to detect the target organism, rather than relying on detecting characterising features of the cells or the microorganisms e.g. by morphological or biochemical markers. Genetic variations may be identified by similar means.

In general, DNA or RNA is identified by hybridisation to one or more oligonucleotides under conditions of stringency sufficient to ensure a low level of non specific binding. Commonly, the hybridising nucleotides are used in pairs as primers in the various forms of in vitro amplification now available, primarily the polymerase chain reaction (PCR) and Strand Displacement Analysis (SDA) but also the Ligase Amplification Reaction (LAR), the Self-Sustained Sequence Replication (3 SR) and the Q-beta replicase amplification system. After amplification the DNA may be further characterised by sequencing, e.g. by the Sanger method. Amplification and sequencing may be combined.

The consistent theme in all detection methods based on nucleic acid amplification is the presence of an initial nucleic acid isolation step, to separate the nucleic acid from materials, e.g. protein, which may interfere in the hybridisation and/or amplification techniques which are used.

A range of methods are known for the isolation of nucleic acids, but generally speaking, these rely on a complex series of extraction and washing steps and are time consuming and laborious to perform.

Classical methods for the isolation of nucleic acids from complex starting materials such as blood or blood products or tissues involves lysis of the biological material by a detergent or chaotrope, possibly in the presence of protein degrading enzymes, followed by several extractions with organic solvents e.g. phenol and/or chloroform, ethanol precipitation, centrifugations and dialysis of the nucleic acids. Not only are such methods cumbersome and time consuming to perform, but the relatively large number of steps required increases the risk of degradation, sample loss or cross-contamination of samples where several samples are simultaneously processed.

Improvements in methods for isolating nucleic acids are thus continually being sought. Methods have been proposed which rely upon the use of a solid phase. In U.S. Pat. No. 5,234,809, for example, is described a method where nucleic acids are bound to a solid phase in the form of silica particles, in the presence of a chaotropic agent such as a guanidinium salt, and thereby separated from the remainder of the sample. WO 91/12079 describes a method whereby nucleic acid is trapped on the surface of a solid phase by precipitation. Generally speaking, alcohols and salts are used as precipitants. Cells from which the nucleic acids are isolated may be first isolated from the sample by filtration, centrifugation or affinity binding to antibodies attached to a solid phase. After cell concentration in this manner, the DNA is then purified from the concentrated cells, often by classical phenol/chloroform extraction methods as discussed above, with their attendant disadvantages.

Other methods, that involve the sequential steps of binding cells to one solid phase, lysis of those cells and subsequent binding of the released nucleic acid to a second solid support, have been described (U.S. Pat. No. 6,255,477). These methods have been further developed such that the cells and the released nucleic acid are bound by the same solid support (WO98/51693 and WO 01/53525).

The inventors have surprisingly found that a much simpler method than that described in WO 98/51693 is also effective. In particular, a separate step of binding released nucleic acid to the solid support is not required and therefore fewer reagents and fewer steps are needed, e.g. a nucleic acid binding buffer is not required.

In this manner, the presence or absence of target cells in a sample, is ascertained by a simple and quick to perform procedure which may take less than 30 minutes.

In a first aspect the present invention therefore provides a method for detecting the presence or absence of a target cell in a sample, said method comprising:

(a) binding cells in said sample to a particulate and mixable solid support;

(b) eluting the cells from the solid support without the use of competitor molecules to disrupt the interaction between the cell and the solid support;

(c) after lysis of said cells, detecting the presence or absence of nucleic acid characteristic of said target cell,

wherein said solid support does not have antibodies or antibody fragments immobilised thereon.

Typically the detection step (c) comprises a nucleic acid amplification method.

The term “cell” is used herein as a convenient way of referring to all prokaryotic (including archaebacteria and mycoplasmas) and eukaryotic cells and other viable entities such as viruses, and sub-cellular components such as organelles. Representative “cells” thus include all types of mammalian and non-mammalian animal cells, plant cells, protoplasts, bacteria, protozoa and viruses. No inference as to the interchangeability of virus specific or cell (i.e. prokaryotic and eukaryotic) specific detection methods should be implied from the use of this term in this way. The “target cell” may also be a particular cell type or a variant of a chosen cell. For instance the target cell may be of the same cell type and be from the same organism as the rest of the cells of the sample but it varies from the rest of the sample in at least one respect, such as a particular mutation in a particular gene.

Preferably the cell is a prokaryotic cell or a eukaryotic cell, more preferably a prokaryotic cell. Most preferred prokaryotic cells are gram negative bacteria (e.g. Bordetella pertussis and Neisseria gonorrhoeae), mollicutes (mycoplasma and ureaplasma, e.g. Mycoplasma pneumoniae) and chlamydia (e.g Chlamydia trachomatis and Chlamydia pneumoniae).

The sample may thus be any material containing nucleic acid within such cells, including for example foods and allied products, clinical and environmental samples. Thus, the sample may be a biological sample, which may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. Representative samples thus include clinical samples taken from the human or animal body such as whole blood and blood-derived products such as plasma or buffy coat, urine, faeces, cerebrospinal fluid or any other body fluids, tissues, cell cultures, cell suspensions etc., and samples obtained by e.g. a swab of a body cavity. Further representative samples include environmental samples such as water samples, e.g. from lakes, rivers, sewage plants and other water-treatment centres or soil samples, or food samples. A preferred sample is urine, respiratory samples and plasma and other blood product components.

The methods are also of notable utility in the analysis of food samples and generally in health and hygiene applications where it is desired to monitor bacterial levels, e.g. in areas where food is being prepared. Milk products for example may be analysed for listeria. Conventional techniques for bacterial isolation using immobilised antibodies have proved to be much less effective than our methods for isolating listeria using non-specific ligands, possibly due to the hydrophobic nature of the immobilised antibody. When the sample is a water sample, the ligand is preferably a nutrient for the microorganisms of interest.

Food samples may be analysed by first homogenising where necessary (if a solid sample) then mixing with a suitable incubation media (e.g. peptone water) and incubating at 37° C. overnight. Food such as cheese, ice cream, eggs, margarine, fish, shrimps, chicken, beef, pork ribs, wheat flour, rolled oats, boiled rice, pepper, vegetables such as tomato, broccoli, beans, peanuts and marzipan may be analysed in this way. The methods of the invention offer particular benefits for the analysis of food samples as these contain a lot of solid material (clumps and fatty particles) which tend to block filters and after centrifugation produce a pellet where the bacteria are packed and not available for lysing or binding to antibodies.

The sample may also include relatively pure or partially purified starting materials, such as semi-pure preparations obtained by other cell separation processes.

The solid supports of use in the methods of the invention are particulate and mixable, i.e. capable of being mixed. To be “mixable” the constituents of the sample and the constituents of the solid support may both be diffused among those of the other during a mixing step, i.e. both components are mobile. Particulate materials, e.g. beads, are advantageous due to their greater binding capacity. Fibres are considered to be a mixable and particulate solid support.

Preferred are materials presenting a high surface area for binding of the cells. Such supports will generally have an irregular surface and may be for example be porous. Conveniently, the support may be made of glass, silica, latex or a polymeric material. Preferably the particles are made of a polymeric material.

Conveniently, a particulate solid support used according to the invention will comprise beads, preferably spherical or substantially spherical beads. The size of the beads is not critical, but they may, for example, have a diameter in the order of at least 1 and preferably at least 2 μm, and have a maximum diameter of preferably not more than 10 and more preferably not more than 6 μm. For example, beads of diameter 2.8 μm and 4.5 μm have been shown to work well.

Monodisperse particles, that is those which are substantially uniform in size (e.g. size having a diameter standard deviation of less than 5%) have the advantage that they provide very uniform reproducibility of reaction. Monodisperse polymer particles produced by the technique described in U.S. Pat. No. 4,336,173 are especially suitable.

Non-magnetic polymer beads suitable for use in the method of the invention are available from Dyno Particles AS (Lillestrøm, Norway) as well as from Qiagen, Pharmacia and Serotec.

However, to aid manipulation and separation, magnetic beads are preferred. The term “magnetic” as used herein means that the support is capable of having a magnetic moment imparted to it when placed in a magnetic field, and thus is displaceable under the action of that field. In other words, a support comprising magnetic particles may readily be removed by magnetic aggregation, which provides a quick, simple and efficient way of separating the particles following the cell and nucleic acid binding steps, and is a far less rigorous method than traditional techniques such as centrifugation which generate shear forces which may disrupt cells or degrade nucleic acids.

Thus, using the method of the invention, the magnetic particles with cells attached may be removed onto a suitable surface by application of a magnetic field e.g. using a permanent magnet. It is usually sufficient to apply a magnet to the side of the vessel containing the sample mixture to aggregate the particles to the wall of the vessel and to pour away the remainder of the sample.

Especially preferred are superparamagnetic particles for example those described by Sintef in EP-A-106873, as magnetic aggregation and clumping of the particles during reaction can be avoided, thus ensuring uniform and nucleic acid extraction. The well-known magnetic particles sold by Dynal AS (Oslo, Norway) as DYNABEADS, are particularly suited to use in the present invention.

Functionalised coated particles for use in the present invention may be prepared by modification of the beads according to U.S. Pat. Nos. 4,336,173, 4,459,378 and 4,654,267. Thus, beads, or other supports, may be prepared having different types of functionalised surface, for example positively or negatively charged, hydrophilic or hydrophobic.

Different cells exhibit different degrees of non-specific binding to different surfaces and supports and it may be advantageous to “titrate” the amount of the solid support (e.g. the number of particles) per volume unit, in order to optimise the cell-binding conditions, and determine the optimum support area, e.g. particle concentration for a given system.

Binding of the cells to the solid support may be achieved in any known or convenient manner. For example, non-specific binding of the cells to the support may be achieved by appropriate choice of the solid support and conditions e.g. the chemical or physical nature of the surface of the solid support, (e.g. hydrophobicity or charge), the pH or composition of the isolation medium etc.

By “non-specific binding” it is meant that a large proportion of the cells (e.g. bacteria) present in the sample are bound by the solid support, both in terms of the proportion of all cells present and the proportion of the types of cell. Thus, preferably at least 30%, more preferably at least 50%, most preferably at least 70 or 80% of the cells in a sample comprising a plurality of cell types will be bound to be solid support. Of course, the percentage of cells in the sample which are bound will depend on the amount of solid support added to the sample and the ratio of cell-binding ligand (if present) to cell. It is assumed for the above percentages that there is an excess of solid support (and cell-binding ligand if applicable) present in the mixture.

Preferably the solid support will be a solid support which is capable of binding most, or all, of the prokaryotic cells in the sample. More preferably, this solid support will be a solid support which is also capable of the preferential binding of prokaryotic cells over eukaryotic cells. Although, some degree of selectivity is present therefore, the binding is still considered to be non-specific. The conditions used during the binding step may influence these capabilities and so the conditions used during the binding step should be selected accordingly. The skilled man is capable of adjusting the binding conditions to optimise them for his needs. Therefore, the non-specific binding step preferably results in the binding of most, or all, of the prokaryotic cells in a sample to the solid support. More preferably, the non-specific binding step results in the binding of most, or all, of the prokaryotic cells in the sample but few, or none, of the eukaryotic cells in the sample.

The nature of the target cells may also play a role and it has, for example, been shown that certain hydrophobic cells may readily bind non-specifically to hydrophobic surfaces, whereas hydrophilic cells may bind to more hydrophilic surfaces. Negatively charged cells such as B-lymphocytes have also been observed to have a high degree of non-specific binding to weakly-positively charged surfaces. Thus, solid supports having appropriately charged surfaces for binding of a desired cell type may be used. Appropriate buffers etc. may be used as media for the cell binding step to achieve conditions appropriate for cell binding, and therefore simply bringing the solid support and the sample into contact in an appropriate medium will result in binding. Conveniently, a buffer of appropriate charge, osmolarity etc. may be added to the sample prior to, simultaneously with, or after contact with the solid support.

Advantageously, non-specific binding of cells may be achieved according to the invention by precipitating the cells onto the support using a precipitant, for example by contacting the cells with the support in the presence of alcohol and salt, e.g. by adding to the sample, a buffer containing alcohol and salt. The use of alcohol and salt in separation and purification procedures such as precipitation is commonplace and any suitable alcohol or salt used in such procedures, may be used according to the present invention. Thus, conveniently the alcohol may be any alkanol, and lower alkanols such as isopropanol and ethanol have been found to be suitable. Other suitable alcohols include methanol and n-butanol.

The salt may be provided by any convenient source e.g. sodium or potassium chloride or acetate, or ammonium acetate. Appropriate concentrations of alcohol and salt may be determined according to the precise system and reagents used. Generally speaking addition of 0.5 to 3 volumes of alcohol e.g. 1 volume, to the sample has been found to be suitable. Conveniently the alcohol may be used at concentrations of 50-100% (w/v). The use of salt concentrations of e.g. 0.1 to 10.0 M, more particularly 0.1 to 7.0 M, e.g. 0.1 to 3.0 M has been found to be suitable, and conveniently the salt may be included, at the above concentrations in the alcohol solution. Thus, a so-called “cell-binding buffer” may be used containing the alcohol and salt at the desired concentrations. Alternatively, the salt and alcohol may be added separately.

The use of alcohol as precipitant for the cells according to the invention is advantageous for use of the method in clinical diagnostic procedures, since the use of alcohol to conserve clinical samples is common. Thus, patient samples may simply be added to an alcohol-containing cell-binding buffer, whereby the samples are conserved and ready for purification of the nucleic acid.

As an alternative to precipitation with salt/alcohol, other precipitants may be used, for example polyethylene glycols (PEGs) or other high molecular weight polymers with similar properties, either alone or in combination with salt and/or alcohol. The concentrations of such polymers may vary depending upon the precise system e.g. polymer and cell type, but generally concentrations from 1 to 50% (w/v), e.g. 2-30% may be used.

Cells with phagocytic activity may be captured by their ability to “bind” or “swallow” a particulate solid phase e.g. beads, and thereby can readily be collected. In this case, the cell-containing sample needs simply to be contacted or incubated with the solid phase under appropriate conditions. This kind of cell capture is not dependent on specific binding.

The solid support may also be provided with moieties which assist in the non-specific binding of cells, for example carbohydrates, proteins or protein fragments or polypeptides which are bound non-specifically by cells. Thus, for example, a solid support coated with carbohydrates binds cells non-specifically through receptors on the cell surface. Techniques for immobilising carbohydrates and other proteins or polypeptides on solid surfaces are well known in the art.

If a ligand is used on the support to effect non-specific binding the ligand is considered a “non-specific ligand”. The “non-specific” ligand will be one which is capable of binding to more than one type of cell preferably to more than 2 or 3, more preferably to more than 5 or 7 e.g. more than 10 or 14 different cell types. There is an interaction between the ligand and its binding partner(s) on the surface of the cell which is responsible for binding, it is not the case that there is simply a general attraction or association between the cells and the solid support, as may be the case when cells bind by precipitation. The non-specific character of the ligand refers not to the fact that it is capable of binding or associating indiscriminately with moieties on the surface cells but that its binding partner(s) is not specific to a certain cell or cell type. The ligand can therefore be considered to be a general binding ligand. As discussed above, although considered non-specific binding, the preferential binding of prokaryotic cells over eukaryotic cells is preferred and so the use of ligands that preferentially bind prokaryotic cells rather than eukaryotic cells, but which bind most, or all, of the prokaryotic cells in the sample are most preferred.

Preferably, non-specific binding does not involve a protein-protein interaction. Therefore, if the non-specific ligand is a protein or protein fragment or a polypeptide, the principle binding partner is not a protein or a part of a protein. Preferably the non-specific ligand is non-proteinaceous. Preferably the non-specific ligand is a carbohydrate.

Suitable carbohydrates include monosaccharides, oligosaccharides (including disaccharides and trisaccharides) and polysaccharides. Suitable monosaccharides include hexoses and pentoses in pyranose and furanose form where appropriate, as well as sugar derivatives such as aldonic and uronic acids and deoxy or amino sugars, anhydro sugars and sugar alcohols. Suitable monosaccharides may be exemplified by mannose (e.g. D-mannose), galactose (e.g. D-galactose), glucose (e.g. D-glucose), fructose, fucose (e.g. L-fucose), N-acetyl-glucosamine, N-acetyl-galactosamine, rhamnose, galactosamine, glucosamine (e.g. D-glucosamine), galacturonic acid, glucuronic acid, N-acetylneuraminic acid, methyl D-mannopyranoside (mannoside), C-methyl-glucoside, galactoside, ribose, xylose, arabinose, saccharate, mannitol, sorbitol, inositol, glycerol and derivatives of these monomers. Of these, mannose, galactose, anhydrogalactose and fucose are preferred.

Particularly preferred are oligosaccharides and polysaccharides which are polymers of monosaccharide monomers, for example polymers incorporating the monosaccharide monomers discussed above and their derivatives.

Oligosaccharides comprise 2 to 12, preferably 4 to 8, covalently linked monosaccharide units which may be the same or different and which may be linear or branched, preferably branched, e.g. oligomannosyl having 2 to 6 units, maltose, sucrose, trehalose, cellobiose, and salicin, particularly maltose. A method for production of oligosaccharides is described in Pan et al. Infection and Immunity (1997), 4199-4206.

Polysaccharides comprise 13 or more covalently linked monosaccharide units which may be the same or different and which may be linear or branched, preferably branched. Suitable polysaccharides will be rich in mannose, galactose, anhydrogalactose, glucose and/or fructose e.g. galactomannan polysaccharide (referred to herein as GUM 1) (Sigma G-0753) which is believed to be a straight chain polymer of mannose with one galactose branch on every fourth mannose.

Further polysaccharides include Gum Arabic (Sigma G 9752) believed to be a branched polymer of galactose, rhamnose, arabionse and glucuronic acid and Gum Karaya (Sigma G 0503) believed to be a partially acetylated polymer of galactose, rhamnose and glucuronic acid.

Polysaccharides which are made up of mannose and galactose sub-units are a preferred type of ligand and a further example is guar (Sigma, G1429) which has a β 1,4 linked linear mannose backbone chain with a galactose side unit on approximately every other unit in a 1,6 α linkage. The mannose to galactose ratio is about 1.8:1 to about 2:1. A further preferred type of ligand are polysaccharides which are made up of mannose, galactose and anhydrogalactose sub-units such as the carrageenans.

Sugar derivatives which are suitable ligands include heparin, heparan sulphate and dextran sulphate. Sulphated sugars are a preferred class of sugar derivatives.

Suitable protein ligands include lectins or fragments of derivatives thereof capable of binding to cells non-specifically, as described above. Antibodies or antibody fragments are not considered suitable proteins.

Ligands based on molecules which are nutrients for microorganisms are also useful ligands. Nutrients for microorganisms which may thus be used as non-specific ligands according to the methods of the present invention include vitamins such as nicotinic acid, riboflavin, thiamin, pyridoxine, pantothenic acid, folic acid, biotin and cobamide and iron-chelating molecules/compounds such as hemin, lactoferrin, transferrin, hemoglobin and certain siderophores such as aerobactin, ferrichrome (Sigma F8014), ferrienterochelin, enterobactin and ferrixanine.

Finally, as mentioned above, non-specific cell-binding to solid supports having charged, hydrophobic or hydrophilic surfaces may be achieved by using buffers, often in combination with salt, to achieve pH conditions appropriate for binding. The precise buffers and conditions will vary depending on the type of cell, solid support etc.

Typically, the various components are mixed and simply allowed to stand for a suitable interval of time to allow the cells to bind to the support. The support may then be removed from the solution by any convenient means, which will depend of course on the nature of the support, and includes all forms of withdrawing the support away from the sample supernatant or withdrawing the sample from the support, for example centrifugation, decanting, pipetting etc.

The conditions during this process are not critical, and it has been found convenient, for example, simply to mix the sample with the “cell-binding buffer” in the presence of a solid phase, and allow it to stand at room temperature, e.g. for 5 to 30 minutes, e.g. 20 minutes before separating. As mentioned above, the reaction time is not critical and as little as 5 minutes may be often enough. However, if convenient, longer periods may be used, e.g. 20 minutes to 3 hours, or even overnight. Mixing can be done by any convenient means, including for example simple agitation by stirring, vortexing, pipetting, inverting or with an alternating magnetic field. Also, if desired, higher or lower temperatures may be used, but are not necessary.

Other optional components in the “cell-binding” composition include high molecular weight polymers e.g. PEGs etc., weak uncharged detergents e.g. Triton X-100, NP-40 etc, DNAses and other enzymes, as long as they leave the cells intact. Preferred “cell-binding” compositions are, for example, PBS, citrate buffers and solutions containing Ca²⁺ and Mg²⁺.

Although non-specific binding of cells is preferred according to the invention, it is also possible to use solid supports which have been modified to permit the selective capture of desired cells containing the nucleic acid. Examples of ligands capable of binding specifically to cells include certain siderophores and cyclic molecules such as steroid molecules and signalling molecules.

By specific it is meant that the ligand is only capable of binding to a single cell type or a single species or genus of cells through the specific binding regions of the ligand. This may introduce a degree of selectivity to the isolation of the nucleic acid, since only nucleic acid from a desired target source within a complex mixture may be separated. Thus for example, such a support may be used to separate and remove the desired target cell type etc only from the sample. The preparation of such selective cell capture matrices is well known in the art and described in the literature.

The cells are bound to the solid support and then may be separated from the remainder of the sample by removing the solid support with cells bound thereto or by removing, e.g. by running off, the remainder of the sample. Where the solid support is magnetic, manipulation of the support/cell complex is especially convenient.

Elution involves the disruption of the interaction between the cell and the solid support. As discussed above, this interaction might be via a solid support-bound ligand. According to the invention, elution does not involve the use of competitor molecules to achieve this disruption.

A competitor molecule is a molecule that binds a second molecule or region of a second molecule in such a way that the binding of at least one further molecule to that second molecule or that region of the second molecule is prevented or discouraged. The binding sites for the competitor molecule and the further molecule in the second molecule or region of a second molecule may be the same or overlapping. Alternatively, they might be distinct but stearic constraints mean that the binding of the competitor molecule is to the exclusion, or partial exclusion, of the further molecule. For instance, the sheer size of the competitor molecule and/or the further molecule might be such that the binding of one prevents that other from accessing its binding site even though those sites are not substantially proximate to one another. Alternatively, the two binding regions might be separated from one another in the primary structure of the molecule they are in, but are proximate to one another by virtue of the tertiary conformation that that molecule assumes.

When cells are bound to a solid support by virtue of a ligand immobilised on that solid support the cell/solid support complex is disruptable with a competitor molecule which corresponds to the components of that complex. For instance, the competitor molecule could be the same as the ligand on the solid support or the binding partner on the cell or be a fragment, an analogue or a homologue thereof that retains the ability to function as a competitor molecule. The competitor molecule could also be the same as the regions within the ligand or binding partner that are involved in the binding reaction or be a fragment, an analogue or a homologue thereof that retains the ability to function as a competitor molecule. The competitor molecule may be present as part of a larger molecule. Typically the competitor molecule, or the molecule it is in, will be free in solution. Thus, if binding is occurring between ligand A and binding partner B, the competitor molecule might be A or a fragment, analogue or a homologue thereof. Alternatively the competitor molecule might be B or a fragment, analogue or a homologue thereof. Typically the competitor will be in excess of A or B as appropriate.

The skilled man would be able to devise suitable elution conditions that do not involve competitor molecules. Typically an elution liquid will be used, which is suitable for the cells and solid supports to be used. Optimisation of the elution liquid would not be unduly burdensome. Examples of suitable elution liquids are water, mild alkalic water, aqueous solutions of bovine serum albumin (BSA), and aqueous salt solutions such as sodium chloride, potassium chloride or magnesium chloride. The elution liquids may be buffered with commonly used buffers such as Tris and MOPS. The choice of elution liquid may be influenced by the downstream detection method that will be employed. For instance if PCR is the chosen amplification technique the elution liquid can comprise appropriate levels of magnesium chloride for the PCR reaction. In fact it has been shown that elution can be achieved with reaction buffers suitable for a subsequent amplification reaction and this is a preferred embodiment of the present invention. For instance, if SDA is the chosen amplification reaction the elution liquid can conveniently be SDA reaction buffer and thus the elution product can be used directly in the SDA reaction.

Elution may be performed at room temperature but elution may be assisted by performing the elution step at an elevated temperature, such as 30-45 degrees Celsius. As discussed below, lysis of cells can be achieved by heating the cells and thus there is a temperature window in which elution can be enhanced but lysis does not occur. The position of this window will depend on the cells involved. For instance, viruses and resilient bacteria such as mycobacteria and chlamydia are reasonably heat resistant and so the window is relatively large. For more delicate cells, such as eukaryotic cells, the window will be smaller. As discussed below, it may however be desirous to lyse the isolated cells at this point in the method.

Detection of the target cell is achieved by the detection of sequences characteristic of the target cell using nucleic acid detection techniques typically based on nucleic acid amplification. Many types of amplification reactions are known in the art and, as mentioned above, PCR, SDA, LAR, 3SR and the Q-beta replicase amplification system are common examples. Preferred amplification methods are PCR and SDA and their modifications e.g. the use of nested primers and real time PCR (see e.g. Abramson and Myers, 1993, Current Opinion in Biotechnology, 4: 41-47 for a review of nucleic acid amplification technologies and Walker et al. 1992 Nucleic Acid Research, 20: 1691-1696 for a description of SDA).

The results of the PCR based or other detection step may be detected or visualised by many means, which are described in the art. For example the PCR or other amplification products may be run on an electrophoresis gel e.g. an ethidium bromide stained agarose gel using known techniques. Alternatively, the DIANA system may be used, which is a modification of the nested primer technique. In the DIANA (Detection of Immobilised Amplified Nucleic Acids) system (see Wahlberg et al., Mol. Cell Probes 4: 285 (1990)), the inner, second pair of primers carry, respectively, means for immobilisation to permit capture of amplified DNA, and a label or means for attachment of a label to permit recognition. This provides the dual advantages of a reduced background signal, and a rapid and easy means for detection of the amplified DNA.

Optionally, one or more washing steps may be introduced into the method of the invention. In particular the support-bound cells may undergo at least one washing step after the support-bound cells have been isolated from the sample. Any solution that does not promote the elution of the cells from the solid support and does not promote the destruction of the cells may be used as a wash buffer. Generally speaking, low to moderate ionic strength buffers are preferred e.g. 10 mM Tris-HCl at pH 8.0/10 mM NaCl. Incorporation of BSA into the washing buffer is also an option. Other standard washing media, e.g. containing alcohols, may also be used, if desired, for example washing with 70% ethanol. Washing solutions of 70% ethanol or PBS are preferred. Conveniently, the wash solution and the binding solution will be the same.

Lysis is achieved by physical means, i.e. lytic chemicals are not required. This includes heating, osmotic shock, sonication, freezing and microwave treatment. One or more of these treatments may be used and preferably lysis is achieved by heating the cells for a suitable period of time at a suitable temperature and/or eluting the cells in a hypotonic solution. Preferably, the cells are heated to between 50° C. and 95° C., more preferably to between 55° C. and 85° C. and most preferably to between 60° C. and 80° C. The duration of heating will depend on the temperature to which the cells are to be heated and the cell types involved but typically lysis is achieved by heating for at least 5 mins, preferably for at least 7 mins, and most preferably for at least 10 mins. Preferably the hypotonic solution is water.

Lysis in this simple manner is particularly suitable because there is no requirement to bind released nucleic acid to the solid support.

The eluted cells may be used directly in a nucleic acid detection method, typically in a nucleic acid amplification reaction. Lysis of the cells is necessary in order to access the nucleic acid for amplification but a separate lysis step or a combined elution and lysis step is not required, the initial heating step of an amplification reaction, designed to denature the nucleic acid, can also serve to lyse the cells. In this context lysis is preferably performed by heating the cells to between 80° C. and 100° C., more preferably to between 90° C. and 98° C. and most preferably to between 90° C. and 95° C.

Therefore in a preferred embodiment the present invention provides a method for detecting of the presence or absence of a target cell in a sample, said method comprising:

(a) binding cells in said sample to a particulate and mixable solid support;

(b) eluting the cells from the solid support without the use of competitor molecules to disrupt the interaction between the cell and the solid support;

(c) lysing the eluted cells by heating; and

(d) detecting the presence or absence of nucleic acid characteristic of said target cell,

wherein said solid support does not have antibodies or antibody fragments immobilised thereon.

The elution step, using the elution liquids described above, may be performed entirely or in part at the above-mentioned lysis temperatures and thus elution and lysis can be achieved in a single convenient step. The resulting products can then be used directly in the amplification reaction. This results in a detection method of considerable simplicity and convenience.

Therefore, in a preferred embodiment the present invention provides a method for detecting of the presence or absence of a target cell in a sample, said method comprising:

(a) binding cells in said sample to a particulate and mixable solid support;

(b) eluting the cells from the solid support without the use of competitor molecules to disrupt the interaction between the cell and the solid support at a sufficiently elevated temperature to cause lysis of said cells; and

(c) detecting the presence or absence of nucleic acid characteristic of said target cell,

wherein said solid support does not have antibodies or antibody fragments immobilised thereon.

The various reactants and components required to perform the methods of the invention may conveniently be supplied in kit form. Such kits represent a further aspect of the invention.

At its simplest, this aspect of the invention provides a kit for detecting the presence or absence of a target cell in a sample comprising:

(a) a particulate and mixable solid support wherein said solid support does not have antibodies or antibody fragments immobilised thereon; optionally

(b) means for binding cells to said solid support; optionally

(c) an elution liquid; and optionally

(d) means for detecting the presence or absence of nucleic acid characteristic of said target cell.

The various means (b), (c) and (d) and the solid support may be as described and discussed above, in relation to the method of the invention.

A typical kit may comprise a solid support, e.g. magnetic particles coated with a polysaccharide such as carrageenan or a protein such as lectin, a binding/washing buffer, e.g. PBS and an elution liquid such as SDA reaction buffer.

The optional component (d) may include appropriate primer oligonucleotides sequences for use in the amplification-based detection techniques.

Optionally further included in such a kit may be buffers, salts, polymers, enzymes etc.

A suitable protocol for use with the kit would be as follows, it is assumed that magnetic or magnetisable beads have been chosen as the solid support (a):
combine binding buffer (b) and beads, add an aliquot of a urine sample and mix, e.g. in an Eppendorf tube,
place under the influence of a magnet and allow the bacteria/bead complex to move to the side of the tube,
pipette off and discard the supernatant,
wash the beads and remove supernatant,
add the elution liquid (c) and incubate at 80° C.,
use the magnet to separate the beads from the supernatant and remove an aliquot of the supernatant and use as template in a PCR reaction with primers specific for nucleic acid characteristic of the target cell, optionally provided by component (d).

In a further aspect the present invention provides a method for detecting the presence or absence of a target cell in a sample, said method comprising:

(a) binding cells in said sample to a particulate and mixable solid support;

(b) eluting the cells from the solid support with a simple elution solution;

(c) after lysis of said cells, detecting the presence or absence of nucleic acid characteristic of said target cell,

wherein said solid support does not have antibodies or antibody fragments immobilised thereon.

Elution of the cells is achieved with a simple elution solution. By “simple elution solution” it is meant any solution that achieves elution without the use of competitor molecules to disrupt the interaction between the cell and the solid support, said interaction may be via a solid support-bound ligand. The elution liquids discussed above are all considered to be suitable elution solutions.

The invention will now be described in more detail in the following non-limiting Examples with references to the drawings in which:

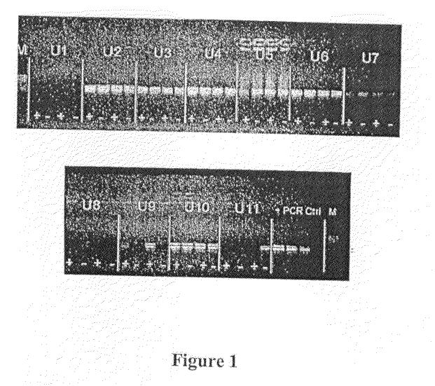

FIG. 1 is a photograph of a gel showing PCR products from Chlamydia trachomatis isolated from specimens previously confirmed positive for Chlamydia trachomatis (confirmed by strand displacement, BDProbeTec) according to the method of Example 1. M: marker; U1-U11: samples; (+)/(−): with/without magnetic mix during initial incubation.

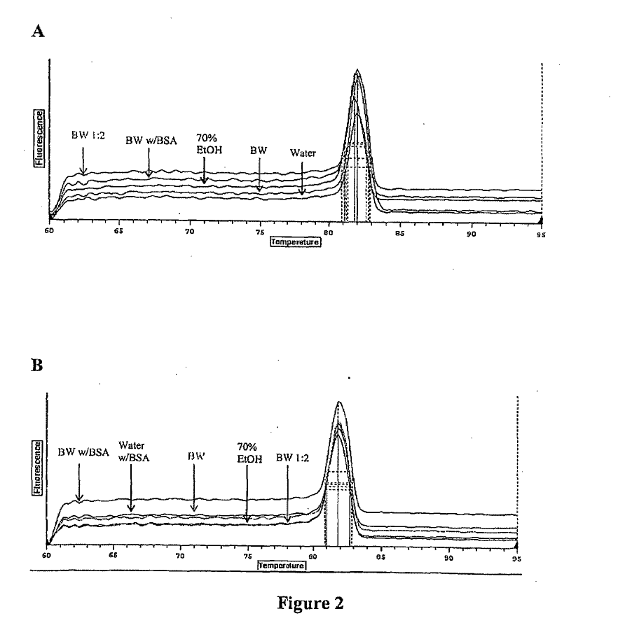

FIG. 2 is a melting analysis of the amplification products from two different samples isolated according to the method of Example 2 under different wash conditions A: sample 2, B: sample 3.

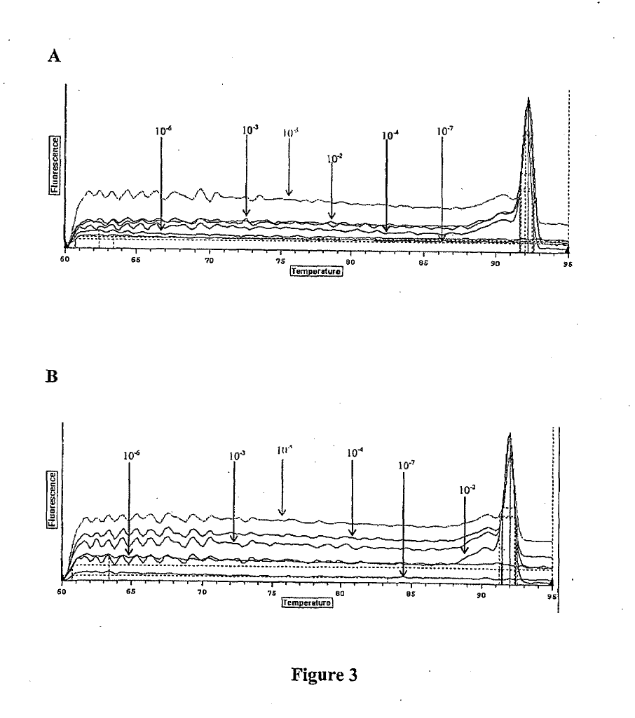

FIG. 3 is a melting analysis of the amplification products from a samples isolated according to the method of Example 3 (A) and samples isolated according to the Bugs n Beads procedure (B) (Genpoint AS, Norway) (Refseth et al., 2004, American Biotechnology Laboratory, June, p 26-28) under different wash conditions.

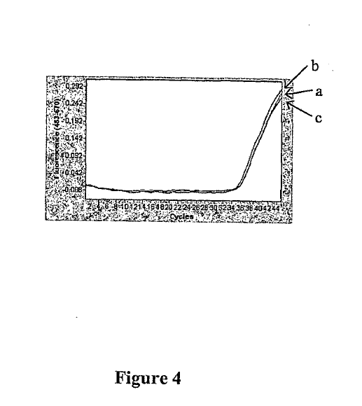

FIG. 4 is a real time PCR analysis of the cDNA obtained from the reverse transcription of the nucleic acid isolated from triplicate samples (a, b and c) of a 10⁻³ dilution of a pooled clinical sample of human Respiratory Syncytial Virus (hRSV).

### EXAMPLE 1

Eleven urine samples, previously determined positive for Chlamydia trachomatis by a commercially available detection system (BDProbeTec, Becton Dickinson), were analyzed using the following protocol for sample preparation combined with PCR analysis.

700 μl urine sample was added manually to 1.5 ml sample tubes in four parallels and loaded into the sample rack carrier of a Tecan Miniprep 75. The remaining part of the isolation procedure was performed automated. BUGS'n BEADS™) BW buffer (Genpoint AS, Norway) and 300 μg magnetic beads (U-version, Genpoint AS, Norway) were added to the samples. Half of the samples were subjected to magnetic mix during the incubation. Following incubation for 15 min, the bacteria/bead complex was immobilized to the side of the tube using a magnetic separator and the supernatant removed. The beads were washed once with 70% EtOH and resuspended in 100 μl water and incubated at 80° C. for 10 minutes to remove residual ethanol. Following incubation the beads were immobilized by magnetic separation, and 15 μl of the supernatant transferred to a PCR plate prefilled with PCR mastermix. The PCR plate was transferred to a MJ Opticon real-time machine for amplification.

PCR amplification was performed as follows. 15 μl of template was used with total volume of 50 μl. Amplification was performed using 20 pmol of the primers Forward: 5′GCAAAAATACACTTGTGGGAGAA3′ and Reverse: 5′GGTGCTCAGACTCCGACATAAT3′ situated in C. trachomatis cryptic plasmid, 0.2 mM dNTP, 1.25 U Hot GoldStar (Eurogentec), 5 mM MgCl₂, 1× Reaction buffer (Eurogentec), SYBR green for detection and 0.02% BSA. The following PCR program was applied, using a MJ Opticon (MJ Research): Initial activation and denaturation at 95° C. for 10 min, then 42 cycles of denaturation at 95° C. for 15 sec, annealing at 65° C. for 45 sec and synthesis at 72° C. for 30 sec. 10 μl of amplified product was loaded onto at 2% agarose gel stained with ethidium bromide.

Results are displayed in FIG. 1. Of the eleven urine samples tested seven were positive for all parallels, one (U9) was positive when using magnetic mix, two were positive for one parallel without magnetic mix samples, and one urine sample (U1) was negative for all parallels. The results show that the isolation can be performed both with and without mixing during the initial incubation.

### EXAMPLE 2

Three urine samples were tested in triplicate with different wash buffers.

Wash Solution Tested:

1. 70% EtOH

2. sdH₂O with 0.05% BSA

3. diluted BW-buffer from the BUGS'n BEADS kit

4. BW-buffer from the BUGS'n BEADS kit

5. BW-buffer from the BUGS'n BEADS kit with 0.05% BSA

Three urine samples previously determined positive for Chlamydia trachomatis by a commercially available detection system (BDProbeTec, Becton Dickinsons) were analyzed using the following protocol for sample preparation combined with PCR analysis. 700 μl of each urine sample was added manually to 1.5 ml sample tubes in four parallels and loaded into the sample rack carrier of a Tecan Miniprep 75. The remaining part of the isolation procedure was performed by an automated system. BUGS'n BEADS™ BW buffer and 300 μg magnetic beads (U-version) were added to the samples. Half of the samples were subjected to magnetic mix during the incubation. Following incubation for 15 min, the bacteria/bead complex was immobilized to the side of tube using a magnetic separator and the supernatant removed. The beads were then washed once with one of wash solutions 1 to 5 and resuspended in 100 μl water and incubated at 80° C. for 10+5 minutes to remove residual ethanol. Following incubation the beads were immobilized by magnetic separation, and 80 μl of the supernatant transferred to a PCR strip and 15 μl transferred manually to a PCR plate preloaded with PCR mastermix. The PCR plate was transferred to a MJ Opticon real-rime PCR machine for amplification

PCR amplification was performed as follows. 15 μl of template was used with total volume of 50 μl. Amplification was performed using 20 pmol of the primers Forward: 5′ GCAAAAATACACTTGTGGGAGAA3′ and 5′GGTGCTCAGACTCCGACATAAT3′ situated in the C. trachomatis cryptic plasmid, 0.2 mM dNTP, 1.25 U Hot GoldStar (Eurogentec), 5 mM MgCl₂ and 1× Reaction buffer (Eurogentec), SYBR green (Eurogentec) for detection and 0.02% BSA. The following PCR program was applied, using a MJ Opticon (MJ Research): initial activation and denaturation at 95° C. for 10 min, then 45 cycles of denaturation at 95° C. for 15 sec, annealing at 65° C. for 45 sec and synthesis at 72° C. for 30 sec. Following amplification a melt curve analysis was performed from 60-95° C., 0.2 C/s.

The results are displayed in FIG. 2. The presence of melting curves from all samples show that different wash solutions can be used following isolation of the bacterial cells and result in successful isolation of DNA.

### EXAMPLE 3

A serial dilution of Mycobacterium abscessus (10⁻² to 10⁻⁷) was prepared and the isolation procedure was performed on a Tecan Miniprep 75 using 70% EtOH as wash solution, followed by analysis with PCR. For comparison the samples were analyzed using the full BUGS'N BEADS protocol performed manually. For robot isolation 30 μl template were used in the PCR whereas 15 μl was used for manual isolated samples.

The protocol for robot isolation was as follows. In parallel, 700 μl of each sample was added manually to 1.5 ml sample tubes. BUGS'n BEADS™ BW buffer and 300 μg magnetic beads (U-version) was then added. Following incubation for 15 min at RT, the bacteria/bead complex was immobilized to the side of tube using a magnetic separator and the supernatant was removed. The beads were washed once with 70% EtOH and resuspended in 100 μl water and incubated at 80° C. for 10+5 minutes to remove residual ethanol. Following incubation the beads were immobilized by magnetic separation and 80 μl of the supernatant was transferred to a PCR strip. 15 μl template for automated and 30 μl template for manual isolation were then transferred to the PCR plate. The PCR plate was then transferred to a MJ Opticon real-rime machine for amplification

PCR amplification was performed as follows. 15 μL/30 μl of template was used with total volume of 50 μl. Amplification was performed using 20 pmol of the primers: Forward: 5′ACCAACGATGGTGTGTCCAT3′ and 5′CTTGTCGAACCGCATACCCT3′ situated in Mycobacterium spp. specific hsp65 gene, 0.2 mM dNTP, 1.25 U Hot GoldStar (Eurogentec), 5 mM MgCl₂ and 1× Reaction buffer (Eurogentec), SYBR green for detection and 0.02% BSA. The following PCR program was applied, using a MJ Opticon (MJ Research): initial activation and denaturation at 95° C. for 10 min, then 45 cycles of denaturation at 95° C. for 15 sec, annealing at 64° C. for 45 sec and synthesis at 72° C. for 30 sec. Following amplification, a melt curve analysis was performed from 60-95° C., 0.2 C/s.

The results are shown in FIG. 3. The presence of a melting curve is indicative of isolation of DNA from the Mycobacteria and so these data are evidence that the isolation protocol may be used with Mycobacterium abscessus. The melting curves are comparable to those achieved with the full BUGS'n BEADS procedure and so the present isolation protocol is comparable with the full BUGS'n BEADS procedure.

### EXAMPLE 4

Urine samples previously determined either positive or negative using the full BUGS' BEADS procedure together with strand displacement amplification (SDA) (BDProbetec, Becton Dickinson) were analysed using the method described in Example 1 on a Tecan Miniprep 75 pipetting robot together with SDA.

The following parameters were tested:
Both C and U version beads of the BUGS'n BEADS kits
70% ethanol as wash buffer
BW-buffer from the BUGS'n BEADS kit as wash buffer
BW-buffer from the BUGS'n BEADS kit with 0.05% BSA as wash buffer
elution by incubation with SDA reaction buffer (BDProbeTec diluent) at RT ° C. for 10 min.
elution by incubation with SDA reaction buffer (BDProbeTec diluent) at 80° C. for 10 min.
elution by incubation with SDA reaction buffer (BDProbeTec diluent) at 80° C. for 5 min followed by RT ° C. for 5 min.

All samples were isolated in parallels, one for testing for C. trachomatis (CT) and one for the amplification control (AC) to visualize any potential inhibition of the stand displacement amplification. The AC was not included for the full BUGS'n BEADs procedure. Following DNA isolation, SDA was performed according to the BDProbetec manual.

The results are displayed in Table 1 below. All previously determined C. trachomatis positive samples displayed positive using the method described in Example 1. Both the C-version and the U version beads of the BUGS'n BEADS kit (Genpoint) gave positive results, demonstrating that different solid supports can be used. All AC were above cut-off for inhibited samples as defined by the ProbeTec kits manufacturer. Positive results for C. trachomatis were obtained regardless of wash buffer used and elution conditions following wash. This shows that elution at room temperature is still effective. This shows that lysis of the isolated cells is not essential.

TABLE 1

| | Methods | | | | | |
|---|---|---|---|---|---|---|
| | FuH BUGS'n BEADS | | New isolation protocol | | | |
| | CT | +/−/≡ | CT | AC | | |
| Samples ID | MOTA | MOTA | MOTA | MOTA | +/−≡ | Final results |
| Sample A - c* | N/A | N/A | 57715 | 30647 | + | + |
| Sample B - c* | N/A | N/A | 35732 | 51721 | + | + |
| Sample C - c* | N/A | N/A | 14565 | 47981 | GZ | unresolved |
| Sample A | 102796 | + | 53924 | 39195 | + | + |
| Sample B | 46973 | + | 80075 | 41182 | + | + |
| Sample D | 47865 | + | 103540 | 33419 | + | + |
| Sample E | 91433 | + | 71678 | 16293 | + | + |
| Sample F | 64717 | + | 85720 | 34172 | + | + |
| Sample G | 59190 | + | 49604 | 33002 | + | + |
| Sample H | 53554 | + | 62662 | 37498 | + | + |
| Sample I | 70958 | + | 88678 | 57646 | + | + |
| Sample J | 50286 | + | 75778 | 35776 | + | + |
| Sample K | 0 | − | 0 | 37348 | − | − |
| Sample C | 0 | − | 0 | 42373 | − | |
| Sample L | 0 | − | 0 | 53826 | − | − |
| Sample M | 0 | − | 3 | 45017 | − | − |
| Sample A 70% 5 min | N/A | N/A | 81273 | 37391 | + | + |
| Sample A 70% 0 min | N/A | N/A | 36866 | 29133 | + | + |
| Sample A | N/A | N/A | 58495 | 51160 | + | + |
| BW w/0.05& BSA 10 min | | | | | | |
| Sample A BW 10 min | N/A | N/A | 58271 | 40551 | + | + |
| Sample A | N/A | N/A | 65290 | 36101 | + | + |
| BW w/0.05% BSA 5 min | | | | | | |
| Sample A BW 5 min | N/A | N/A | 88394 | 50412 | + | + |
| Sample A | N/A | N/A | 9736 | 38131 | GZ | |
| BW w/0.05% BSA 0 min | | | | | | |
| Sample A BW 0 min | N/A | N/A | 24745 | 32813 | + | + |
| Pos ctrl untreated | N/A | N/A | 8904 | 51750 | GZ | |
| Neg ctrl untreated | N/A | N/A | 0 | 28827 | − | − |
| Pos ctrl 1 min | N/A | N/A | 3013 | 30399 | GZ | |
| Neg ctrl 1 min | N/A | N/A | 20 | 37141 | − | − |
| Pos ctrl 3 min | N/A | N/A | 57739 | 24942 | + | + |
| Neg ctrl 3 min | N/A | N/A | 162 | 18639 | − | − |
| Pos ctrl 5 min | N/A | N/A | 75035 | 31523 | + | + |
| Neg ctrl 5 min | N/A | N/A | 191 | 31094 | − | − |
| Pos ctrl 10 min | N/A | N/A | 55967 | 26746 | + | + |
| Neg ctrl 10 min | N/A | N/A | 135 | 37741 | − | − |
| Pos ctrl | N/A | N/A | 44961 | 42726 | + | + |
| Neg ctrl | N/A | N/A | 0 | 37148 | − | − |
| 12228 untreated | N/A | N/A | 910 | 725 | ≡ | − |
| Pos ctrl 20 min | N/A | N/A | 16012 | 23149 | GZ | |
| Neg ctrl 20 min | N/A | N/A | 0 | 31698 | − | − |
| Pos ctrl | N/A | N/A | 40156 | 28473 | + | + |

CT: MOTA value for Chlamydia trachomatis with Strand Displacement Amplification
AC: MOTA value for Amplification Control with Strand Displacement Amplification
≡: Inhibition
GZ—grayzone
*c version beads from the BUGS'n BEADS kit

### EXAMPLE 5

A 10⁻³ diluted sample of human Respiratory Syncytial Virus (hRSV) was analysed in triplicate using the following protocol.

The initial 10⁻³ dilution was made in Copan virus transport medium from pooled clinical sputum samples that were previously determined to be hRSV positive.

100 μl of the diluted hRSV sample was added manually to 1.5 ml samples tubes in three parallels. 150 μg of magnetic beads (U-version) were then added. Following incubation for 15 min at RT, the bacteria/bead complex was immobilized to the side of the tube using a magnetic separator and the supernatant was removed. The beads were washed once with 10 mM Tris and resuspended in 50 μl water and incubated at 80° C. for 10 minutes. Following incubation, the beads were immobilized by magnetic separation and 45 μl of the supernatant was transferred to a new tube ready for the reverse transcription reaction.

The reverse transcription reaction was performed using the following reaction conditions and a LightCycler 480. In a final volume of 20 μl there was combined 9 μl of template (supernatant from the above procedure), hexamer primer (0.02 μg/μl), reaction mix including RT enzyme (20 U/μl; RevertAid™ M-MuLV RT, Fermentas) and Ribonuclease Inhibitor (2 U/μl; RiboLock™, Fermentas) The reaction mixture was then incubated at 25° C. for 5 min and then 42° C. for 60 min, following which, inactivation occurred at 70° C. for 10 min.

FRET PCR detection was then performed in accordance with Whiley et al. J. Clin. Microbiol. 2002, 40(12): 4418-4422 and using a LightCycler 480. The primers used were RS upp (5′-GCCAAAAAATTGTTTCCACAATA-3′) and RS low (5′-TCTTCATCACCATACTTTTCTGTTA-3′). The probes used were RSV-LC1 (5′-GTTGTTCTATAAGCTGGTATTGATGCA-3′fluorescein) and RSV-LC2 (Cy5-GGAATTCACATGGTCTACTACTGACTGT-3′phosphate). 18 μl mastermix (1× reaction buffer, 3.5 mM MgCl₂, 200 μM dNTP and Taq polymerase (Hot GoldStar) 0.025 U/μl, 400 nM of each primer and 200 nM of each of the two probes) was used with 2 μl template (the product from the reverse transcription reaction) and subjected to an initial incubation at 95° C. for 10 min, followed by 45 cycles of 95° C. for 10 sec, 55° C. for 45 sec and 72° C. for 15 sec.

Results are show in FIG. 4.

1. A method for detecting the presence or absence of a target cell in a sample, said method comprising:
(a) binding cells in said sample to a particulate and mixable solid support;
(b) eluting the cells from the solid support without the use of competitor molecules to disrupt the interaction between the cell and the solid support;
(c) after lysis of said cells, detecting the presence or absence of nucleic acid characteristic of said target cell,
wherein said solid support does not have antibodies or antibody fragments immobilised thereon.

2. The method of claim 1 wherein said cell is a prokaryotic cell or a eukaryotic cell.

3. The method of claim 1 wherein said cell is a gram negative bacteria, a mollicute or chlamydia.

4. The method of claim 3 wherein said cell is selected from the group consisting of Bordetella pertussis, Neisseria gonorrhoeae, Mycoplasma pneumoniae, Chlamydia trachomatis and Chlamydia pneumoniae.

5. The method of claim 1 wherein the sample is an environmental sample, a clinical sample or a food sample.

6. The method of claim 1 wherein the solid support comprises beads.

7. The method of claim 6 wherein the beads are magnetic beads.

8. The method of claim 1 wherein the binding of the cells in the sample to the solid support is by non-specific binding.

9. The method of claim 8 wherein the solid support is brought into contact with the sample in the presence of a medium that allows the non-specific binding of the cells in the sample to the solid support.

10. The method of claim 9 wherein the medium that allows the non-specific binding of cells to the solid support contains a precipitant.

11. The method of claim 10 wherein the precipitant is an alcohol and/or a salt and/or a polyethylene glycol.

12. The method of claim 11 wherein the alcohol is selected from the group consisting of isopropanol, ethanol, methanol and n-butanol.

13. The method of claim 11 wherein the salt is selected from the group consisting of sodium acetate, potassium acetate, sodium chloride, potassium chloride and ammonium acetate.

14. The method of claim 1 wherein said binding of the cells to the solid support is assisted by a non-specific cell binding moiety immobilised on the solid support.

15. The method of claim 14 wherein the non-specific cell binding moiety is a polysaccharide comprising mannose, galactose, anhydrogalactose, glucose, fructose and/or derivatives thereof.

16. The method of claim 15 wherein the polysaccharide is selected from the group consisting of GUM 1, Gum Arabic, Gum Karaya, guar, carrageenan, heparin, heparan sulphate and dextran sulphate.

17. The method of claim 9 wherein the medium that allows the non-specific binding of the cells in the sample to the solid support is PBS, citrate buffers, solutions containing Ca²⁺ or solutions containing Mg²⁺.

18. The method of claim 1 further comprising a step wherein the cells bound to the solid support are separated from the remainder of the sample by removing the solid support with cells bound thereto from the remainder of the sample.

19. The method of claim 1 wherein the elution is performed in an elution liquid selected from the group consisting of water, mild alkalic water, aqueous solutions of bovine serum albumin and aqueous solutions of sodium chloride, potassium chloride and/or magnesium chloride.

20. The method of claim 19 wherein the elution liquid contains Tris or MOPS.

21. The method of claim 1 wherein the presence or absence of nucleic acid characteristic of said target cell is detected by a nucleic acid amplification based technique.

22. The method of claim 1 wherein lysis of the cells is by heating and/or by osmotic shock.

23. The method of claim 1 wherein elution of the cells from the solid support and lysis is done in a single step.

24. The method of claim 23 wherein lysis is by elution in a hypotonic solution and/or at an elevated temperature.

25. The method of claim 1 wherein the cells are used directly in a nucleic acid detection method.

26. The method of claim 1 further comprising one or more washing steps.

27. A kit for detecting the presence or absence of a target cell in a sample comprising:
(a) a particulate and mixable solid support wherein said solid support does not have antibodies or antibody fragments immobilized thereon; optionally
(b) means for binding cells to said solid support; optionally
(c) an elution liquid; and optionally
(d) means for detecting the presence or absence of nucleic acid characteristic of said target cell.
